(19) United States
(12) Patent Application Publication
(10) Pub. No.: US 2021/0395804 A1
(43) Pub. Date: Dec. 23, 2021

(54) SENSITIVE AND MULTIPLEXED DETECTION OF NUCLEIC ACIDS AND PROTEINS FOR LARGE SCALE SEROLOGICAL TESTING

(71) Applicant: California Institute of Technology, Pasadena, CA (US)

(72) Inventors: Long CAI, Pasadena, CA (US); Chee Huat (Linus) ENG, Pasadena, CA (US); James J. PARK, Pasadena, CA (US); Yujing YANG, Pasadena, CA (US); Michal POLONSKY, Pasadena, CA (US)

(21) Appl. No.: 17/352,162

(22) Filed: Jun. 18, 2021

Related U.S. Application Data
(60) Provisional application No. 63/041,591, filed on Jun. 19, 2020.

(51) Int. Cl. C12Q 1/6837; C12Q 1/6841; G01N 33/68; G01N 33/543

(52) U.S. Cl. CPC C12Q 1/6837; G01N 33/54326; G01N 33/68; C12Q 1/6841

(57) ABSTRACT

This disclosure herein sets forth embodiments to provide a serological test to detect target analytes that can scale to up to 10,000 or more samples in a single run. This disclosure herein sets forth methods to allow for unique barcoding by using a multi-level barcode scheme that is modular and enables easy detection of multiple analytes in samples.

### CROSS-REFERENCE TO RELATED APPLICATIONS

This application claims the benefit of U.S. Provisional Application Ser. No. 63/041,591, filed Jun. 19, 2020, the entirety of which is incorporated herein by reference.

### FIELD OF INVENTION

The invention disclosed herein generally relates to methods and systems for large scale serological testing for analytes.

### BACKGROUND

Pandemics require rapid, accurate and large-scale testing to provide consistent surveillance and reporting on potential outbreaks. Current methods for RNA detection can require purification, reverse-transcription, and PCR amplification to detect the viral RNA. Similarly, current methods to detect proteins can require purification, and the use of labeled antibodies to detect the proteins. Currently, these tests can require cumbersome purification protocols and enzymatic reactions to simultaneously measure multiple parameters such as proteins, antibodies, nucleic acids, or small molecules. Even with automation, high throughput detection is routinely hindered by reagent cost, labor, and instrument availability.

### SUMMARY

The present disclosure provides methods for detecting one or more target analytes in one or more samples for large scale serological testing of a population. This disclosure sets forth processes and using the same, and other solutions to problems in the field.

In some embodiments, a method is disclosed herein for detecting one or more target analytes in one or more samples comprising providing one or more samples. In some embodiments, the method comprises contacting each sample with one or more primary affinity reagents, wherein each primary affinity reagent specifically binds to one or more target analytes in the one or more samples, under conditions that permit binding. In some embodiments, the method comprises contacting one or more ID reagents to the one or more target analytes, wherein each ID reagent comprises: one or more ID barcodes; and one or more capture reagents capable of binding to the one or more target analytes or one or more primary affinity regents. In some embodiments, the method comprises pooling the primary affinity reagents or the ID reagents bound to the one or more target analytes. In some embodiments, the method comprises detecting the one or more target analytes and the one or more ID barcodes on each ID reagent or primary affinity reagent.

In some embodiments, a method is disclosed herein for detecting one or more target analytes in one or more biological samples comprising providing one or more samples. In some embodiments, the method comprises contacting each sample with one or more primary affinity reagents, wherein each primary affinity reagent binds one or more target analytes in the one or more samples, under conditions that permit binding. In some embodiments, the method comprises contacting the one or more samples with one or more ID reagents to one or more target analytes, wherein each ID reagent comprises: one or more capture reagents, each capture reagent specific for one or more target analytes; and optionally, at least two or more oligonucleotide handles, wherein one of the oligonucleotide handles identifies a specific primary affinity reagent, and wherein one or more of the oligonucleotide handles identify a sample. In some embodiments, the method comprises capturing the ID reagents on a substrate for visualization. In some embodiments, the method comprises detecting one or more target analytes, a handle identifying a specific primary affinity reagent, and one or more handles identifying the sample on each primary affinity reagent or ID reagent.

In some embodiments, a method is disclosed herein for detecting one or more target analytes in one or more samples comprising providing one or more samples. In some embodiments, the method comprises contacting each sample with one or more primary affinity reagents, wherein each primary affinity reagent binds one or more target analytes in the one or more samples, under conditions that permit binding. In some embodiments, the method comprises contacting the one or more samples with one or more ID reagents, wherein each ID reagent comprises: one or more capture reagents, each capture reagent specific for one or more target analytes; and optionally, one or more oligonucleotide handles. In some embodiments, the method comprises capturing the one or more primary affinity reagents or one or more ID reagents on a substrate for visualization. In some embodiments, the method comprises detecting one or more target analytes, a handle identifying a specific primary affinity reagent, one or more handles identifying the sample on each primary affinity reagent, ID reagent, or any combination thereof.

In some embodiments, the method comprises diagnosing a subject by detecting one or more target analytes according to any of the previous embodiments. In certain embodiments, the method is used to diagnosing a subject by detecting one or more target analytes according to any of the previous embodiments.

In some embodiments, the method comprises treating a subject by monitoring a subject, or having monitored a subject, administering, or having administered, a therapeutic agent to the subject. In certain embodiments, the method is used to treat a subject by monitoring a subject, or having monitored a subject, administering, or having administered, a therapeutic agent to the subject.

In some embodiments, any of the previous method are useful to provide a serological test to detect target analytes that can scale to up to 10,000 or more samples in a single run

### DETAILED DESCRIPTION

The following description is presented to enable one of ordinary skill to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1: Large scale testing of SARS-CoV2 RNA and antibodies using multifunctional ID beads and sequential hybridization. Samples from 10,000 patients in 96 well plates (step 1a) are hybridized with multifunctional ID beads (step 1b) that contain different capture reagents and barcodes (steps 2 and 3). In step 4, two level barcoding allows 96×96=9216 samples to be uniquely barcoded. In steps 5 and 6, the ID beads are pooled together and captured on a glass slide and imaged on a microscope by sequential hybridization. The sequential images in step 6 are decoded and the results outputs the status of each sample for both antibodies and RNA (step 7).

FIG. 2: Detection of synthetic SARS-CoV2 RNA with single molecule sensitivity. Synthetic SARS-CoV2 RNAs were captured on a glass coverslip and hybridized using FISH probes. Individual dots correspond to single RNA molecules. The number of dots detected correspond to 80% of the number of RNAs injected into the flow cell.

FIG. 3: SARS-CoV2 RNA captured on functionalized ID beads were visualized by single molecule FISH.

FIG. 4: Capturing and detecting antibodies on ID beads. ID Beads are functionalized with β-galactosidase protein and sample barcode oligonucleotides. In the presence of an anti-β-galactosidase antibody, secondary antibody signal is detected (top middle panel). Without primary antibodies, only faint bead autofluorescence is observed (bottom middle panel).

FIG. 5: Titration of β-galactosidase primary antibodies. The detection is linear over a range of antibody concentration and sensitive down to 1 ng.

FIG. 6: IgG signals on saliva of a COVID-19 positive patient sample. The signals correspond to an average fluorescence on 200 ID beads functionalized with either spike protein, RBD, β-galactosidase, or no antigens. β-galactosidase and no antigen levels are due to bead autofluorescence.

FIG. 7: Multiplexing quantification of soluble protein in fluid samples using sequential hybridization. (A) ID beads are conjugated with capture reagent antibodies and 3 kinds of oligonucleotide handles. Each handle can bind to 4 different DNA bridges. Those DNA bridges are divided into 2 pairs, pair A and pair B. By combining DNA bridge with different concentration ratios (8 levels for pair A and 12 levels for pair B), ID beads can be barcoded into 96 barcodes using 4 DNA bridges. (B) For convenience, ID beads with different capture reagent antibodies are barcoded with DNA bridges targeting the first oligonucleotide handles in the first round. Barcoded ID beads are then pooled together, split, and incubated with patient samples together with DNA bridges targeting the second oligo handle. Targeting analytes and plate coordinate ID are obtained in (7.1. and 7.2.) ID beads from the same plate are pooled together and incubated with DNA bridges binding to the third handle to obtain plate ID barcoding (7.3). After 3 rounds of barcoding, ID beads are all pooled together to incubate with fluorescence detection reagent (7.4). ID beads are then captured on a coverslip for detection antibody, quantification, and DNA bridge sequentially hybridization (7.5) and (7.6).

FIG. 8: A representative image showing the intensity difference between DNA bridge pairs. ID beads conjugated with oligonucleotide handles and capture reagent antibodies were pipetted into a 96 well plate containing titrations of DNA bridges for a 1 hour incubation, following the previously described strategy. The ID beads were then captured on a glass coverslip, and FISH readouts were flown in sequentially to visualize the abundances of each DNA bridge. Red circles show the contour of the beads. The fluorescence intensity differs for each bead in each hybridization round, corresponding to the individual ratiometric barcode of each ID bead.

FIG. 9: Calculated fluorescence ratio of 96 well ratiometric barcoding of one handle. ID beads conjugated with oligonucleotide handles and capture reagent antibodies were pipetted into 96 well plate containing titrations of DNA bridges, and incubated for 1 hour following the previously described strategy. ID beads were then captured on a glass coverslip, and fluorescent FISH readouts were flown in sequentially to visualize the abundances of all four DNA bridges, as shown in (A). Histograms showing the normalized ratio of fluorescent intensities between DNA bridge pairs. The first ratiometric pair creates 8 unique levels (top histogram) and the second creates 12 levels (bottom histogram). (B) Same data as in A, shown as a scatter plot of the 96 unique barcodes created by the four DNA bridges. Each dot is one ID bead, and the location of each bead in the plot indicates a unique combination of the four bridges. The different colors correspond to different fields of view that were imaged in the same experiment. Intensity values of ID beads with the same barcode detected in different fields of view fall very close together, showing that the imaging is uniform over a large area.

FIG. 10: Using machine learning to distinguish barcoded clusters. To facilitate an automated assignment of barcodes to ID beads, we used machine learning to cluster the ID beads according to their normalized intensities in the 8×12 barcode space. Each bead was assigned to a cluster which is the effective barcode. The clusters were then traced back to the normalized ratiometric intensities allowing for correct classification of the beads.

FIG. 11: (A) shows the fluorophore intensity of beads treated with IL-1b standards under fluorescent microscopy. (B) shows that the concentration of IL-1b in standards is correlated with bead fluorescent intensities.

### DEFINITIONS

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges. Oligonucleotides can be of various lengths. In particular embodiments, oligonucleotides can range from about 2 to about 1000 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to form a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal. In some embodiments, a “probe” may be a small molecule.

As used herein, the term “binding sites” refer to a portion of a probe where other molecules may bind to the probe. In certain embodiments, the binding sites of a probe bind to another molecule through a non-covalent interaction.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As used herein, the term “dot” refers to the single molecules that fluoresce as they are excited by light of a particular frequency. In certain embodiments, a nucleic acid or protein, or combination thereof hybridized to a oligonucleotide probe or protein probe or combination thereof produces a excitation “dot” when excited by a particular frequency.

As disclosed herein, the term “DNA bridge” or “bridge” or “DNA bridge probe” refers to probes that are complementary to other probes that may hybridize or bind to the complementary probes, the other probes are not covalently linked 5′ to 3′ to each other. In certain embodiments, the term “bridge probe” uses the definition and techniques of Lohman et al. Efficient DNA ligation in DNA-RNA hybrid helices by Chlorella virus DNA ligase. Nucleic Acid Research, 2014, vol. 42 No. 3 1831-1844, incorporated by its entirety.

As used herein, the term “antibody” refers to any macromolecule that would be recognized as an antibody by those of skill in the art. In some embodiments, an antibody includes any form of an antibody other than the full length form that would be recognized by an antibody fragment by those of skill in the art.

As used herein, the term “bind” refers to two or more proteins, oligonucleotides, small molecules, antibodies, and combinations thereof to interact to form larger complexes. In certain embodiments “bind” refers to the hybridization of oligonucleotides with RNA, DNA, oligonucleotides, or combinations thereof.

As defined herein, the term “analytes” refers to nucleic acids, RNA, DNA, proteins, antibodies, antibody fragments, hormones, carbohydrates, lipid molecules, small molecules, biologically active molecules, and any combination thereof. In certain embodiments, a person of skill would recognize “analytes” or “disease markers” refers to a substance or measurable parameter that can be used to identify the presence of a condition or assess the status of known disease.

As defined herein, the term “capture reagent” refers to a nucleic acid, antibody, antibody fragment, small molecule, or any combination thereof capable of hybridizing or binding to a target analyte.

As defined herein, the term “ID bead” or “bead” refers to a particle that may be conjugated to one or more oligonucleotides, antibodies, antibody fragments, proteins, small molecules or any combination thereof.

As defined herein, the term “ID reagent” refers to ID beads, nucleic acids, antibodies, antibody fragments, proteins, small molecules, or any combination thereof that serves to hybridize or bind to a target analyte and aids in identifying the sample. In certain embodiments, the ID reagents comprises ID beads.

As defined herein, the term “oligonucleotide handles” or “handles” refers to a single stranded DNA sequence with a 3′ overhang.

As disclosed herein, the term “biologically active molecule” refers to molecules that exert a direct physiological effect on an organism. For instance, milk proteins, the proteins themselves, as well as any peptides that result from proteolytic degradation are known to affect the immune system.

This disclosure herein sets forth embodiments to provide a serological test to detect target analytes that can scale to up to 10,000 or more samples in a single run. The methods disclosed herein are able to use existing automated pipelines for large scale serological tests of a population. The methods disclosed herein eliminate typical enzymatic and purification steps by concentrating all samples into a single coverslip or flowcell for imaging on a single instrument for a fast turnaround time.

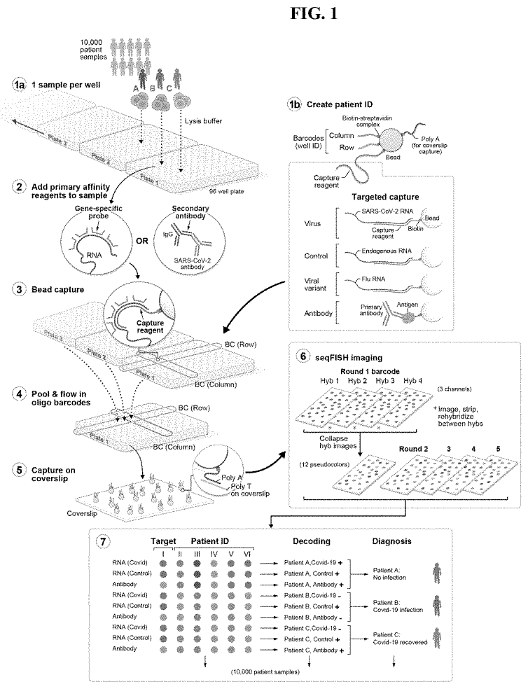

This disclosure herein sets forth methods to screen up to 10,000 or more patient samples in 96 well formats, allowing for unique barcoding by using a multilevel barcoding scheme. FIG. 1 describes, a two level barcode scheme comprising one “well” barcode (based on column and row identification of a 96 well plate) and another “plate” barcode. A total of 9216 unique pairs of barcodes can be used based on 96 individual well barcodes and at least 96 individual plate barcodes (96×96=9216). A three level barcoding scheme provides 96³=884,736 unique barcodes. A four level barcoding scheme provides 96⁴=74,934656 unique barcodes.

This disclosure herein sets forth methods to use and make multi-functionalized beads that are modular and enable easy detection of multiple markers in samples. Different types of beads are constructed to capture a panel of analytes. This pool of ID beads can then be barcoded uniquely in each well and plate. The beads are further used to concentrate nucleic acids, proteins, or combinations thereof to increase detection signals. This disclosure sets forth methods herein to make and use multi-functionalized beads that are magnetic, allowing handling the beads and transferring them between samples without purification kits.

### SARS-CoV2

In some embodiments, the method comprises detecting a virus in a sample. In certain embodiments, the method comprises detecting the severe acute respiratory syndrome coronavirus 2 (SARS-CoV2). In certain embodiments, the method comprises decoding sample barcodes in a pooled sample to determine the presence of SARS-CoV2 RNA and antibodies from each sample. In certain embodiments, the method comprises determining the presence of antibodies derived from a patient in response to infection by SARS-CoV2 infection. In certain embodiments, the method comprises detecting the SARS-CoV2 RNAs with single molecule sensitivity.

In some embodiments, the method comprises detecting the presence of the SARS-CoV2 virus against other viruses in a population. In certain embodiments, the method comprises using probes capable of distinguishing viral transcripts versus host derived genomic transcripts, host derived antibodies or antibody fragments, or combinations thereof to determine the infection status of a patient.

In some embodiments, the method comprises detecting antibodies in the same patient samples. In certain embodiments, the method comprises detecting an IgG specific for the SARS-CoV2 spike protein. In certain embodiments, the method comprises detecting an IgG for the SARS-CoV2 receptor binding domain.

In some embodiments, a method is disclosed herein for detecting one or more target analytes in one or more samples comprising providing one or more samples. In some embodiments, the method comprises contacting each sample with one or more primary affinity reagents, wherein each primary affinity reagent specifically binds to one or more target analytes in the one or more samples, under conditions that permit binding. In some embodiments, the method comprises contacting one or more ID reagents to the one or more target analytes, wherein each ID reagent comprises: one or more ID barcodes; and one or more capture reagents capable of binding to the one or more target analytes or one or more primary affinity regents. In some embodiments, the method comprises pooling the primary affinity reagents or the ID reagents bound to the one or more target analytes. In some embodiments, the method comprises detecting the one or more target analytes and the one or more ID barcodes on each ID reagent or primary affinity reagent.

In some embodiments, a method is disclosed herein for detecting one or more target analytes in one or more biological samples comprising providing one or more samples. In some embodiments, the method comprises contacting each sample with one or more primary affinity reagents, wherein each primary affinity reagent binds one or more target analytes in the one or more samples, under conditions that permit binding. In some embodiments, the method comprises contacting the one or more samples with one or more ID reagents to one or more target analytes, wherein each ID reagent comprises: one or more capture reagents, each capture reagent specific for one or more target analytes; and optionally, at least two or more oligonucleotide handles, wherein one of the oligonucleotide handles identifies a specific primary affinity reagent, and wherein one or more of the oligonucleotide handles identify a sample. In some embodiments, the method comprises capturing the ID reagents on a substrate for visualization. In some embodiments, the method comprises detecting one or more target analytes, a handle identifying a specific primary affinity reagent, and one or more handles identifying the sample on each primary affinity reagent or ID reagent.

In some embodiments, a method is disclosed herein for detecting one or more target analytes in one or more samples comprising providing one or more samples. In some embodiments, the method comprises contacting each sample with one or more primary affinity reagents, wherein each primary affinity reagent binds one or more target analytes in the one or more samples, under conditions that permit binding. In some embodiments, the method comprises contacting the one or more samples with one or more ID reagents, wherein each ID reagent comprises: one or more capture reagents, each capture reagent specific for one or more target analytes; and optionally, one or more oligonucleotide handles. In some embodiments, the method comprises capturing the one or more primary affinity reagents or one or more ID reagents on a substrate for visualization. In some embodiments, the method comprises detecting one or more target analytes, a handle identifying a specific primary affinity reagent, one or more handles identifying the sample on each primary affinity reagent, ID reagent, or any combination thereof.

In some embodiments, the method comprises diagnosing a subject by detecting one or more target analytes according to any of the previous embodiments.

In some embodiments, the method comprises treating a subject by monitoring a subject, or having monitored a subject, administering, or having administered, a therapeutic agent to the subject.

In some embodiments, the method comprises detecting one or more target analytes. In some embodiments, the target analytes in the sample are selected from proteins, modified proteins, transcripts, RNA, DNA loci, exogenous proteins, exogenous nucleic acids, hormones, carbohydrates, small molecules, biologically active molecules, and combinations thereof. In some embodiments, the target analytes comprises nucleic acids, proteins, or combinations thereof.

In some embodiments, the method comprises placing each biological sample into a single well of a multi-well plate.

In some embodiments, the method comprises lysing each sample with a lysis buffer to obtain target nucleic acids, target proteins, target analytes, or combinations thereof.

In some embodiments, the primary affinity reagent specifically hybridizes or binds one or more targets in the sample. In some embodiments, the primary affinity reagent comprises a specific oligonucleotide, protein, antibody, small molecule, or combination thereof. In some embodiments, the primary affinity reagent comprises an oligonucleotide. In some embodiments, the one or more primary affinity reagents comprises an oligonucleotide complementary to one or more target nucleic acids in the sample. In some embodiments, the primary probe comprises an antibody. In some embodiments the antibody comprises an antibody specific to a target protein that identifies the target protein. In some embodiments, the primary affinity reagent comprises a protein. In some embodiments, the primary affinity reagent comprises a small molecule.

In some embodiments, the primary affinity reagent comprises a nucleic acid complementary to the target nucleic acids in the sample. In some embodiments, the primary affinity reagent comprises a protein capable of binding to one or more target nucleic acids or proteins or any combination thereof in the sample. In some embodiments, the primary affinity reagent comprises a protein-nucleic acid complex capable of binding to target nucleic acids or proteins in the sample. In some embodiments, the one or more primary affinity reagents comprises an oligonucleotide complementary to one or more target nucleic acids in the sample. In some embodiments, the one or more primary affinity reagents comprises a protein. In some embodiments, the one or more primary affinity reagents comprises a small molecule. In some embodiments, the one or more primary affinity reagents comprises an oligonucleotide, protein, an antibody, a small molecule, or any combination thereof.

In some embodiments, the primary affinity reagent comprises a nucleic acid complementary to the nucleic acids in the patient sample. In some embodiments, the sequence complementarity is at least 60%, 65%, 70%, 75%, 80%, 85%, 90%, 91%, 92%, 93%, 94%, 95%, 96%, 97%, 98%, 99%, or 100%.

In some embodiments, the capture reagent hybridizes or binds the primary affinity reagent or target analyte. In some embodiments, the capture reagent comprises an oligonucleotide, protein, antibody, small molecule, or combination thereof. In some embodiments, each capture reagent comprises an oligonucleotide, protein, antibody, small molecule, or combination thereof. In certain embodiments, the capture reagent is capable of binding to the target analytes. In certain embodiments, the capture reagent is capable of binding to the primary affinity reagent.

In some embodiments, the capture reagent comprises a nucleic acid complementary to the nucleic acids in the patient sample. In some embodiments, the sequence complementarity is at least 60%, 65%, 70%, 75%, 80%, 85%, 90%, 91%, 92%, 93%, 94%, 95%, 96%, 97%, 98%, 99%, or 100%.

In some embodiments, the ID reagents serve to hybridize or bind to target analytes and aids in identifying the sample. In some embodiments, the ID reagents are selected from ID beads, nucleic acids, antibodies, antibody fragments, proteins, small molecules, or any combination thereof. In certain embodiments, the ID reagents comprises ID beads. In certain embodiments, the ID beads may range in size from 5 nm to 5 mm. In certain embodiments, the ID beads comprise ceramic, gold, polystyrene, semiconductor quantum dots, or magnetic particles. In certain embodiments, each ID bead is magnetic.

In some embodiments, each oligonucleotide handle is conjugated to the ID bead by its amino terminus to a carboxyl moiety on the bead, and wherein the oligonucleotide has a 3′ overhang. In some embodiments, the method comprises one or more oligonucleotide handles that identifies a specific primary affinity reagent. In some embodiments, the method comprises one or more oligonucleotide handles that identify a sample. In some embodiments, the method comprises one or more oligonucleotide handles that identifies a specific primary affinity reagent, and one or more oligonucleotide handles identify a sample.

In some embodiments, the method further comprises isolating the one or more ID reagents. In some embodiments, the method further comprises isolating the one or more ID reagents before or after any step of the method. In some embodiments, the one or more ID reagents comprise patient ID barcodes or plate ID barcodes or any combination thereof. In some embodiments, the one or more patient ID barcodes comprise oligonucleotides that identify a well in a multi-well plate.

In some embodiments, the patient ID barcodes are added to the primary affinity reagent or ID reagents after the primary affinity reagent or ID reagent contacts the target analytes in the sample. In some embodiments, the patient ID barcodes are added to the primary affinity reagent or ID reagents before the primary affinity reagent or ID reagent contacts the target analytes in the sample.

In some embodiments, the plate ID barcodes are added to the primary affinity reagents or ID reagents after the primary affinity reagents or ID reagents contact the target analytes in the sample. In some embodiments, the plate ID barcodes are added to the primary affinity reagents or ID reagents after the primary affinity reagents or ID reagents contact the target analytes in the sample.

In some embodiments, the method further comprises pooling ID beads from wells of a single plate and transferring the pool into an individual well of a multi-well plate. In some embodiments, the method further comprises pooling primary affinity reagent from wells from a single plate and transferring the pool into an individual well of a multi-well plate. In certain embodiments, the plate barcode is added after the pooled ID beads are transferred to an individual well of a multi-well plate.

In some embodiments, the method comprises hybridizing each ID bead with a first DNA bridge probe to bind the first handle, prior to contacting the sample. In some embodiments, the method comprises pooling each ID bead after hybridizing the first DNA bridge probe to the first handle, and splitting into a multi-well plate to hybridize the second DNA bridge probe to a second handle, prior to contacting with a sample. In some embodiments, the method comprises pooling each primary affinity reagent after hybridizing the first DNA bridge probe to the first handle, and splitting into a multi-well plate to hybridize the second DNA bridge probe to a second handle, prior to contacting with a sample. In some embodiments, the method comprises pooling ID beads after hybridizing the first DNA bridge probe to the first handle, splitting into a multi-well plate to hybridize a second DNA bridge probe to a second handle, and splitting from each well in the multi-well plate to hybridize a third DNA bridge probe to a third handle, prior to contacting with the sample. In some embodiments, the method comprises pooling each primary affinity reagent after hybridizing the first DNA bridge probe to the first handle, splitting into a multi-well plate to hybridize a second DNA bridge probe to a second handle, and splitting from each well in the multi-well plate to hybridize a third DNA bridge probe to a third handle, prior to contacting with the sample.

In some embodiments, the method comprises a first DNA bridge probe hybridized to a first oligonucleotide handle to detect one or more target analytes, a handle identifying a specific primary affinity reagent, one or more handles identifying the sample on each primary affinity reagent, ID reagent, or any combination thereof. In some embodiments, the method comprises a second DNA bridge probe hybridized to the first DNA bridge probe to detect one or more target analytes, a handle identifying a specific primary affinity reagent, one or more handles identifying the sample on each primary affinity reagent, ID reagent, or any combination thereof. In some embodiments, the method comprises a third DNA bridge probe hybridized to the second DNA bridge probe to detect one or more target analytes, a handle identifying a specific primary affinity reagent, one or more handles identifying the sample on each primary affinity reagent, ID reagent, or any combination thereof.

In some embodiments, the ID reagent comprises one or more barcodes identifying a row number, column number, or any combination thereof of a well in a multi-well plate, wherein each well corresponds to one or more samples.

In some embodiments, the ID reagent comprises a barcode that binds to the ID bead through a covalent interaction. In some embodiments, the ID reagent comprises a barcode that binds to the ID bead through a non-covalent interaction. In certain embodiments, the ID reagent comprises a barcode that binds to the ID bead that includes, but is not limited to a streptavidin-biotin system, oligonucleotide hybridization, his-tag and his-tag binding reagent, and any combinations thereof. In certain embodiments, the ID reagent comprises a barcode that binds to the ID bead through a biotin-streptavidin interaction. In certain embodiments, the barcode comprises biotin and the patient ID bead comprises streptavidin. In certain embodiments, the barcode comprises streptavidin and the ID bead comprises biotin.

In some embodiments, the one or more ID beads comprises one or more substrate binding moieties. In some embodiments, the substrate binding moiety hybridizes, binds, conjugates, or any combination thereof to a substrate to allow for detection. In certain embodiments, the method comprises contacting the ID beads to a substrate, wherein the substrate binds the substrate binding moiety on the ID bead.

In some embodiments, the substrate is coated on a surface. In some embodiments, the substrate is coated on a cover slip. In some embodiments, the substrate is coated on a flow cell. In some embodiments, the substrate is coated on substance already coated to the surface.

In some embodiments, the substrate binding moiety comprises polymers that can be crosslinked to a substrate. In certain embodiments, the substrate binding moiety comprises an oligonucleotide. In certain embodiments, the substrate comprises an oligonucleotide binding molecule. In certain embodiments, the substrate binding moiety comprises an oligonucleotide binding molecule. In certain embodiments, the substrate comprises an oligonucleotide. In certain embodiments, the substrate binding moiety comprises a poly A oligonucleotide sequence. In certain embodiments, the substrate binding moiety comprises a poly T oligonucleotide sequence. In certain embodiments, the substrate comprises a poly T oligonucleotide sequence. In certain embodiments, the substrate comprises a poly A oligonucleotide sequence. In certain embodiments, the substrate binding moiety comprises a streptavidin molecule. In certain embodiments, the substrate binding moiety comprises a biotin molecule. In certain embodiments, the substrate comprises a streptavidin molecule. In certain embodiments, the substrate comprises a biotin molecule.

In some embodiments, a substrate moiety forms a covalent linkage between a cover slip or flow cell and the ID beads. In certain embodiments, the substrate moiety which comprises, but is not limited to N-hydroxysuccinimide (NHS), maleimide, iodoacetyl, 1-ethyl-3-(3-dimethylamino) propyl carbodiimide (EDC), hydrazines, alkyne, azide, DBCO, tetrazine, trans-cyclooctane, amino, thiol, or any combinations of above-mentioned reagents.

In some embodiments, the substrate binding moiety binds the substrate via magnetic force, gravitic force, frictional force, or any combination thereof. In certain embodiments, the substrate binds the substrate binding moiety via magnetic force, gravitic force, frictional force, or any combination thereof. In some embodiments, the method comprises trapping the ID beads on glass for detection. In some embodiments, the trapping is performed by embedding beads within a hydrogel, trapping beads within microwells, magnetic interactions, and directly coating glass slides with adherence particles, or any combinations thereof.

In some embodiments, the methods comprise multiplexing immuno-detection or RNA quantitation or combinations thereof from a sample. In certain embodiments, the methods comprise multiplexed immuno-detection or nucleic acid quantitation or combinations thereof. In certain embodiments, the methods comprise multiplexed immuno-detection of one or more proteins, antibodies, antibody fragments, analytes, nucleic acids, or combinations thereof.

In some embodiments, polymerase chain reaction is performed on the nucleic acid sample. In certain embodiments, each patient ID bead comprises a capture reagent that is complementary to the PCR product. In certain embodiments, one or more biotin modified primers are used during PCR, allowing for the direct functionalization of the PCR product on the patient ID beads. In certain embodiments, the captured PCR products are then analyzed by seqFISH or FISH as described in any of the embodiments described herein.

In some embodiments, the method comprises screening at least 1,000; 2,000; 3000; 4000; 5000; 6000; 7000; 8000; 9000; 10000; 15,000; 20,000; 30,000; 40,000; 50,000, 60,000; 70,000; 80,000; 90,000; 100,000; 200,000; 300,000; 400,000; 500,000; 600,000; 700,000; 800,000; 900,000; or 1,000,000 samples. In some embodiments, the method of any of the preceding embodiments comprises up to 10000; 15,000; 20,000; 30,000; 40,000; 50,000, 60,000; 70,000; 80,000; 90,000; 100,000; 200,000; 300,000; 400,000; 500,000; 600,000; 700,000; 800,000; 900,000; or 1,000,000 samples.

In some embodiments, the method comprises screening at least 1, 2, 3, 4, 5, 6, 7, 8, 9, 10, 15, 20, 25, 30, 35, 40, 45, 50, 60, 70, 80, 90, or 100 target analytes. In some embodiments, the method of any of the preceding embodiments comprises up to 5, 6, 7, 8, 9, 10, 15, 20, 25, 30, 35, 40, 45, 50, 60, 70, 80, 90, 100, 500, 1000, 5000, or 10,000 target analytes.

### Sequential Hybridization, FISH, and seqFISH

In some embodiments, the method comprises fluorescence detection. In some embodiments, the method comprises fluorescence detection or other methods of detection. In some embodiments, the method comprises sequential hybridization to detect target analytes. In some embodiments, the method comprises sequential hybridization to detect oligonucleotide handles. In some embodiments, the method comprises sequential hybridization to detect the primary affinity reagents.

Certain techniques for analyzing the biological samples are known. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the seqFISH method comprises performing a first contacting step that comprises contacting a sample, wherein the sample comprises one or more cells, or wherein the sample is processed from one or more cells, or wherein the sample comprises a plurality of nucleic acids with a first plurality of detectably labeled oligonucleotides, each of which targets a nucleic acid in the sample and is labeled with a detectable moiety, so that the composition comprises at least: (i) a first oligonucleotide targeting a first nucleic acid in the sample and labeled with a first detectable moiety; and (ii) a second oligonucleotide targeting a second nucleic acid in the sample and labeled with a second detectable moiety. In some embodiments, the method comprises imaging the cell sample after the first contacting step so that interaction by the first plurality of oligonucleotides with their targets is detected. In some embodiments, the method comprises repeating the contacting and imaging steps, each time with a plurality of detectably labeled oligonucleotides wherein at least one target nucleic acid contacted by multiple pluralities of detectably labeled oligonucleotides is targeted with a different detectable moiety labeling of oligonucleotides in at least one of the pluralities. In some embodiments, the method comprises optionally, performing additional rounds of contacting and imaging prior or in between or after steps (a)-(e) for error correction with block codes.

In some embodiments, the seqFISH method comprises performing a first contacting step that comprises contacting a cell sample, wherein the sample comprises one or more cells, or wherein the sample, comprises a plurality of target proteins or target nucleic acids, with a first plurality of detectably labeled proteins or oligonucleotides or combinations thereof, each of which targets a target protein or target nucleic acid and is labeled with a detectable moiety, so that the composition comprises at least: (i) a first protein or oligonucleotide or combination thereof, targeting a first target protein or target nucleic acid in the sample and labeled with a first detectable moiety; and (ii) a second protein or oligonucleotide or combination thereof, targeting a second target protein or target nucleic acid in the sample and labeled with a second detectable moiety. In some embodiments, the seqFISH method comprises (b) imaging the sample after the first contacting step so that interaction by proteins or oligonucleotides or combinations thereof, of the first plurality of detectably labeled proteins or oligonucleotides or combinations thereof, with their targets is detected. In some embodiments, the seqFISH method comprises repeating the contacting and imaging steps, each time with a plurality of detectably labeled proteins or oligonucleotides or combinations thereof, wherein at least one target protein or target nucleic acid in the sample is contacted by multiple pluralities of detectably labeled proteins or oligonucleotides or combinations thereof, is targeted with a different detectable moiety labeling of proteins or oligonucleotides or combinations thereof in at least one of the pluralities. In some embodiments, the method comprises optionally, performing additional rounds of contacting and imaging prior or in between or after steps (a)-(e) for error correction with block codes.

In some embodiments, the seqFISH method comprises performing a first contacting step that comprises contacting a sample, wherein the sample comprises one or more cells, or wherein the sample comprises a plurality of target proteins or target nucleic acids, with a first plurality of intermediate proteins or intermediate oligonucleotides or combinations thereof, each of which: (i) targets a target protein or target nucleic acid in the sample and is optionally labeled with a detectable moiety; and (ii) optionally, comprises an overhang sequence after hybridization with the target; so that the composition comprises at least: (i) a first intermediate protein or oligonucleotide or combination thereof, targeting a target first protein or target nucleic acid in the plurality of target proteins or target nucleic acids and optionally labeled with a first detectable moiety; and (ii) a second intermediate protein or oligonucleotide or combination thereof, targeting a second target protein or target nucleic acid in the plurality of target proteins or target nucleic acids and optionally labeled with a second detectable moiety. In some embodiments, the seqFISH method comprises contacting the first plurality of intermediate proteins or intermediate oligonucleotides with a first plurality of detectably labeled proteins or oligonucleotides or combinations thereof comprising at least: (i) a first detectably labeled protein or oligonucleotide or combination thereof, targeting a set of the intermediate proteins or oligonucleotides or combination thereof; and (ii) optionally, a second detectably labeled protein or oligonucleotide or combination thereof, targeting a set of the intermediate proteins or oligonucleotides or combination thereof. In some embodiments, the seqFish method comprises imaging the sample after contacting the first plurality of intermediate proteins or intermediate oligonucleotides with one or more detectably labeled proteins or oligonucleotides or combinations thereof, so that the interaction of the intermediate protein or intermediate oligonucleotide with their targets is detected. In some embodiments, the seqFISH method comprise repeating the contacting and imaging steps, each time with a plurality of detectably labeled proteins or oligonucleotides or combinations thereof that target intermediate proteins or oligonucleotides or combinations thereof bound to target proteins or target nucleic acids, wherein at least one-intermediate protein or oligonucleotide or combination thereof is targeted with a different detectable moiety labeling of proteins or oligonucleotides or combinations thereof in at least one of the pluralities. In some embodiments, the seqFISH method comprises optionally, performing additional rounds of contacting the sample intermediate proteins or oligonucleotides or combinations thereof. In some embodiments, the seqFISH method comprises optionally, performing additional rounds of contacting and imaging prior or in between or after steps (a)-(e) for error correction with block codes.

In some embodiments, the seqFISH method comprises contacting the sample with a plurality of intermediate proteins or intermediate oligonucleotides or combinations thereof, each of which: (i) targets a target protein or target nucleic acid in the sample and is optionally labeled with a detectable moiety; and (ii) optionally, comprises an overhang sequence after hybridization with the target. In certain embodiments, the seqFISH method comprises optionally, imaging the cell so that interaction between the intermediate oligonucleotides with their targets is detected.

In some embodiments, the seqFISH method comprises an error correction round performed by selecting from block codes such as Hamming codes, Reed-Solomon codes, Golay codes, or any combination thereof.

In some embodiments, the seqFISH method comprises removing readout probes by using stripping reagents, wash buffers, photobleaching, chemical bleaching, and any combinations thereof.

### Stabilization

In some embodiments, the primary affinity reagents are stabilized by methods selected from enzyme ligation, chemical ligation, UV crosslinking with or without oligo splint probes, hybridization of splint probes, crosslinking through a matrix, and chemical crosslinking, or any combination thereof. In certain embodiments, the enzymes used for enzyme ligation are selected from T4 Ligase, T7 Ligase, quick ligase, and ampligase. In certain embodiments, the chemical ligation is selected from comprise amine-phosphate, diamine, and thiol ligation. In certain embodiments, the crosslinking through the matrix comprises a hydrogel made from polyacrylamide or agarose. In certain embodiments, the chemical crosslinking for stabilization is selected from paraformaldehyde, glutaraldehyde, and reversible crosslinkers such as DSP (dithiobis succinimidyl propionate).

In some embodiments, the capture reagents are stabilized by methods selected from enzyme ligation, chemical ligation, UV crosslinking with or without oligo splint probes, hybridization of splint probes, crosslinking through a matrix, and chemical crosslinking, or any combination thereof. In certain embodiments, the enzymes used for enzyme ligation are selected from T4 Ligase, T7 Ligase, quick ligase, and ampligase. In certain embodiments, the chemical ligation is selected from comprise amine-phosphate, diamine, and thiol ligation. In certain embodiments, the crosslinking through the matrix comprises a hydrogel made from polyacrylamide or agarose. In certain embodiments, the chemical crosslinking for stabilization is selected from paraformaldehyde, glutaraldehyde, and reversible crosslinkers such as DSP (dithiobis succinimidyl propionate).

### Flurophores

In some embodiments, the method comprises detecting target analytes, ID barcodes, primary affinity reagents, and any combination thereof with fluorophores. In some embodiments, the method comprises detecting target analytes, ID barcodes, primary affinity reagents, handles and any combination thereof with fluorophores.

In some embodiments, the fluorophore is any fluorophore deemed suitable by those of skill in the arts.

In some embodiments, the fluorophores include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the fluorphores include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium(II), Texas Red, TSQ, Umbelliferone, or Yellow fluorescent protein.

In some embodiments, the fluorophores include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the fluorophores comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the fluorophore comprises a nanomaterial. In some embodiments, the fluorophore is a nanoparticle. In some embodiments, the fluorophore is or comprises a quantum dot. In some embodiments, the fluorophore is a quantum dot. In some embodiments, the fluorophore comprises a quantum dot. In some embodiments, the fluorophore is or comprises a gold nanoparticle. In some embodiments, the fluorophore is a gold nanoparticle. In some embodiments, the fluorophore comprises a gold nanoparticle.

### Washes

In some embodiments, the method of any of the preceding embodiments, comprises optionally washing the sample after each step. In certain embodiments, the sample is washed with a buffer that removes non-specific hybridization reactions. In certain embodiments, formamide is used in the wash step. In certain embodiments, the wash buffer is stringent. In certain embodiments, the wash buffer comprises 10% formamide, 2×SSC, and 0.1% triton X-100s.

### Handles, Id Barcodes, and Plate Barcodes

In some embodiments, the handles of any of the preceding embodiments comprises oligonucleotides that are at least 5 nucleotides in length. In some embodiments, the handles of any of the preceding embodiments comprises oligonucleotides that are at least 10 nucleotides in length In some embodiments, the handles of any of the preceding embodiments comprises oligonucleotides that are at least 11 nucleotides in length. In some embodiments, the handles of any of the preceding embodiments comprises oligonucleotides that are at least 12 nucleotides in length. In some embodiments, the handles of any of the preceding embodiments comprises oligonucleotides that are at least 13 nucleotides in length. In some embodiments, the handles of any of the preceding embodiments comprises oligonucleotides that are at least 14 nucleotides in length. In some embodiments, the handles of any of the preceding embodiments comprises oligonucleotides that are at least 15 nucleotides in length. In some embodiments, the handles of any of the preceding embodiments comprises oligonucleotides that are at least 16 nucleotides in length. In some embodiments, the handles of any of the preceding embodiments comprises oligonucleotides that are at least 17 nucleotides in length. In some embodiments, the handles of any of the preceding embodiments comprises oligonucleotides that are at least 18 nucleotides in length. In some embodiments, the handles of any of the preceding embodiments comprises oligonucleotides that are at least 19 nucleotides in length. In some embodiments, the handles of any of the preceding embodiments comprises oligonucleotides that are at least 20 nucleotides in length. In some embodiments, the handles of any of the preceding embodiments comprises oligonucleotides that are at least 21 nucleotides in length. In some embodiments, the handles of any of the preceding embodiments comprises oligonucleotides that are less than 30, 50, 100, 200, 250, 500, 750, or 1000 nucleotides in length.

In some embodiments, the ID barcode of any of the preceding embodiments comprises oligonucleotides that are at least 10 nucleotides in length. In some embodiments, the ID barcode of any of the preceding embodiments comprises oligonucleotides that are at least 11 nucleotides in length. In some embodiments, the ID barcode of any of the preceding embodiments comprises oligonucleotides that are at least 12 nucleotides in length. In some embodiments, the ID barcode of any of the preceding embodiments comprises oligonucleotides that are at least 13 nucleotides in length. In some embodiments, the ID barcode of any of the preceding embodiments comprises oligonucleotides that are at least 14 nucleotides in length. In some embodiments, the ID barcode of any of the preceding embodiments comprises oligonucleotides that are at least 15 nucleotides in length. In some embodiments, the ID barcode of any of the preceding embodiments comprises oligonucleotides that are at least 16 nucleotides in length. In some embodiments, the ID barcode of any of the preceding embodiments comprises oligonucleotides that are at least 17 nucleotides in length. In some embodiments, the ID barcode of any of the preceding embodiments comprises oligonucleotides that are at least 18 nucleotides in length. In some embodiments, the ID barcode of any of the preceding embodiments comprises oligonucleotides that are at least 19 nucleotides in length. In some embodiments, the ID barcode of any of the preceding embodiments comprises oligonucleotides that are at least 20 nucleotides in length. In some embodiments, the ID barcode of any of the preceding embodiments comprises oligonucleotides that are at least 21 nucleotides in length. In some embodiments, the ID barcode of any of the preceding embodiments comprises oligonucleotides that are less than 30, 50, 100, 200, 250, 500, 750, or 1000 nucleotides in length.

In some embodiments, the plate barcode of any of the preceding embodiments comprises oligonucleotides that are at least 10 nucleotides in length. In some embodiments, the plate barcode of any of the preceding embodiments comprises oligonucleotides that are at least 11 nucleotides in length. In some embodiments, the plate barcode of any of the preceding embodiments comprises oligonucleotides that are at least 12 nucleotides in length. In some embodiments, the plate barcode of any of the preceding embodiments comprises oligonucleotides that are at least 13 nucleotides in length. In some embodiments, the plate barcode of any of the preceding embodiments comprises oligonucleotides that are at least 14 nucleotides in length. In some embodiments, the plate barcode of any of the preceding embodiments comprises oligonucleotides that are at least 15 nucleotides in length. In some embodiments, the plate barcode of any of the preceding embodiments comprises oligonucleotides that are at least 16 nucleotides in length. In some embodiments, the plate barcode of any of the preceding embodiments comprises oligonucleotides that are at least 17 nucleotides in length. In some embodiments, the plate barcode of any of the preceding embodiments comprises oligonucleotides that are at least 18 nucleotides in length. In some embodiments, the plate barcode of any of the preceding embodiments comprises oligonucleotides that are at least 19 nucleotides in length. In some embodiments, the plate barcode of any of the preceding embodiments comprises oligonucleotides that are at least 20 nucleotides in length. In some embodiments, the plate barcode of any of the preceding embodiments comprises oligonucleotides that are at least 21 nucleotides in length. In some embodiments, the plate barcode of any of the preceding embodiments comprises oligonucleotides that are less than 30, 50, 100, 200, 250, 500, 750, or 1000 nucleotides in length.

### Ratiometric Labeling

In some embodiments, the method of any of the preceding claims comprises ratiometric barcodes. In some embodiments, the method comprises using ratiometric barcodes to distinguish samples and plates. In some embodiments, the method comprises titrating one or more DNA bridge probes at different ratios for each oligonucleotide handle. In some embodiments, the method comprises four DNA bridges per handle, the DNA bridges are separated into two pairs of oligonucleotides. In some embodiments, the method comprises preparing one or more ratios within each pair of DNA bridge as a barcode. In some embodiments, the method comprises using the first pair of DNA bridges to titrate a handle with a first set of ratios. In some embodiments, the method comprises using the second pair of DNA bridges to titrate a handle with second set of ratios. In some embodiments, the first set of ratios comprises 8 levels of titration. In some embodiments, the second set of ratios comprises 12 levels of titration. In some embodiments, each level of titration comprises ratios of a pair of oligonucleotides.

### Concentration Determination

In some embodiments, the method comprises determining the target analyte concentration in a sample. In certain embodiments, concentration is determined by measuring signal intensity from the signal and correlating intensity to target analyte concentration. In certain embodiments, the analyte concentration is determined by preparing a standard curve with a known concentration of analyte in a 96 well format. In certain embodiments, the analyte concentration is determined by measuring the fluorophore intensity of the primary affinity reagent or ID reagent compared to the standard curve.

Having described the embodiments in detail, it will be apparent that modifications, variations, and equivalent embodiments are possible without departing the scope of the defined in the appended claims. Furthermore, it should be appreciated that all examples in the present disclosure are provided as non-limiting examples.

The following non-limiting methods and examples are provided to further illustrate the embodiments disclosed herein. It should be appreciated by those of skill in the art that the techniques disclosed in the methods and examples that follow represent approaches that have been found to function well in practice, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the embodiments.

### Method 1

As shown in FIG. 1, 96 unique ID beads with unique “well barcodes” were constructed and pipetted into different wells of a 96 multi-well plate, where each well held a different sample. Beads with the same “well barcodes” are pipetted into the same wells of different multi-well plates.

The beads were then incubated with the patient samples for 1-5 hours to bind the RNA or antibodies in each well. To uniquely barcode the beads in each well and plate, a “plate” barcode biotin-oligo was added to each well and conjugated to the beads. The beads now contained a unique pair of “well” and “plate” barcode, as well as the RNA or antibodies captured by them.

All of the beads were then pooled and bound to a glass coverslip (1.5). The beads were then hybridized or bound with fluorescence in situ hybridization (FISH) probes or secondary antibodies to determine the presence of the molecular species on each bead. At the same time, the well and plate barcodes corresponding to a particular sample were decoded through multiple rounds of hybridization by seqFISH techniques (1.6). In ten rounds of hybridization using three color imaging, 3¹⁰=59,049 different barcodes were resolved in a ten hour experiment on an automated microscope with fluidics. Tabulating the individual beads and the corresponding molecular signals on these beads allowed us to determine the abundance of RNA and antibodies present in each patient sample (1.7).

### Method 2

### A Ratio-Metric Method for Efficient Barcoding of Magnetic Beads

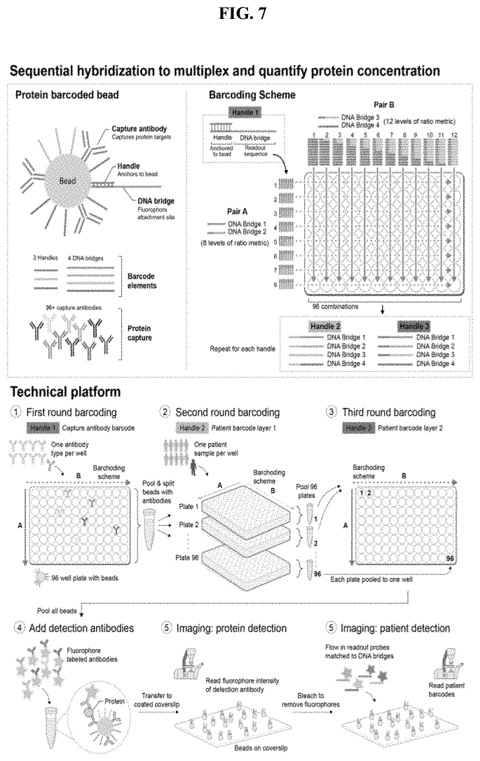

As shown in FIG. 7, three different oligonucleotide handles (DNA or LNA) were conjugated to carboxyl coated magnetic beads. Capture antibody targeting specific analyte were also conjugated to beads at the same time. To barcode a sample, DNA bridges with a 3′ end reverse complement to handle sequence and a 5′ end overhang, allowing read out by seqFISH were incubated with beads.

For each handle, four DNA bridges were used that were separated into two pairs of oligonucleotides. The ratio within each pair of DNA bridge was used as a barcode. For convenience, one pair used to form a DNA bridge had 8 levels of titration ratios (0:7, 1:2, 2:5, 3:4, 4:3, 5:2, 6:1, 7:0). Similarly, another pair of oligonucleotides used formed twelve levels of titration ratios. The two barcoding systems were combined to form 8×12=96 distinguishable ratiometric barcodes, marking beads with 96 barcoding possibilities. Using this method, three handles can provide 96×96×96=884,736 barcodes.

For easier barcode decoding, one handle is used for capture antibody labeling, two handles are used for patient labeling. Each ratio can be read out by hybridizing probes against one of the four bridge oligonucleotides. The intensity ratio between two readouts provides the ratiometric barcode. In total, the twelve readouts were used to readout all 884,736 barcodes.

Using this strategy, 96 analyte targets from 96×96=9216 patient samples were uniquely multiplexed. For analyte barcoding, beads conjugated with the same antibody were barcoded the same. One 96 multi-well plate of different capture antibodies was barcoded according to this scheme. After the capture antibody on the beads was uniquely barcoded, the ID beads were pooled together, mixed, and split into 96 well plates containing uniquely barcoded DNA bridges that bind to the second oligonucleotide handles. Beads in each well were further split into 96 ratio-metric plates to bind to barcode third oligo handle. After all procedures, 96×96=9216 patient barcoded beads with 96 analyte capture antibodies were generated.

Each patient barcoded analyte bead pool was incubated with a patient sample to allow analytes to bind to capture antibodies. To visualize the analyte concentration, 96 corresponding analyte detection antibody pools were incubated with the beads. A detection antibody with biotin was visualized using fluorophore conjugated streptavidin. Beads were then pooled together and captured on a glass coverslip. The first round of imaging read out streptavidin fluorophore intensity to construct the concentration of analyte on each bead. The same areas were photobleached and then FISH (fluorescence in situ hybridization) probes were flown through a flow cell in order to quantify the intensity of DNA bridge pairs on each bead.

With four rounds of three color imaging, (8×12)³ different barcodes were resolved in a three hours experiment on an automated microscope with fluidics. Fish probe intensity ratios between DNA bridge pairs were decoded for all three handles.

### EXAMPLES

### Example 1

### Designed Probes Specific for SARS-CoV2

As shown in Table 1, probes were designed that hybridize against only SARS-CoV2 RNA and not to any other coronavirus family strains such as SARS-CoV1, MERS, human coronavirus 229E, OC43, HKU1, and NL63.

The probes were able to distinguish between the COVID-19 RNA genome as well as COVID-19 RNA transcripts. A set of probes were designed against the Orflab region of the COVID-19 genome as well as another set against the N and S coding regions which appear in transcribed RNA fragments. These two probe sets allow us to measure abundances of the genomic RNA as well as transcribed RNA to report on the activity of the virus in addition to the presence of the virus.

| Probe | Sequence |
|---|---|
| 1 | GCCAGAGATGTCACCTAAATCAAC |
| | ATCTGGTGATG |
| 2 | CCAGCTATAAAACCTAGCCAAATG |
| | TACCATGGCCA |
| 3 | GAGACAACTACAGCAACTG |
| | GTCATACAGCAAAGCA |
| 4 | GTCGTCTTCATCAAATTTGCAGCA |
| | GGATCCACAAG |
| 5 | CTGAAGGAGTAGCATCCTTGATTT |
| | CACCTTGCTTC |
| 6 | GGAGTGAGGCTTGTATCGGTATC |
| | GTTGCAGTAGCG |
| 7 | AACACCCTTGGAGAGTGCTAGTTG |
| | CCATCTCTTTT |
| 8 | GCAGCAACGAGCAAAAGGTGTGA |
| | GTAAACTGTTAC |
| 9 | GAACGGCATTTCCAGCAAAGCCA |
| | AAGCCTCATTAT |
| 10 | CAGTGTCTGTACTCAATTGAGTTG |
| | AGTACAGCTGG |
| 11 | CGTCGATTGTGTGAATTTGGACAT |
| | GTTCTTCAGGC |
| 12 | CGCTAGTAGTCGTCGTCGGTTCAT |
| | CATAAATTGGT |
| 13 | GCGCAGTAAGGATGGCTAGTGTA |
| | ACTAGCAAGAAT |
| 14 | CGGTAATAGTACCGTTGGAATCTGCC |
| | ATGGCTAAA |
| 15 | CAAGCTAAAGTTACTGGCCATAAC |
| | AGCCAGAGGAA |
| 16 | CCACCGGTGATCCAATTTATTCTGT |
| | AAACAGCAGC |
| 17 | CTGAGCCACATCAAGCCTACAAGA |
| | CAAGCCATTGC |
| 18 | ATGGAACGCGTACGCGCAAACAG |
| | TCTGAAAGAAGC |
| 19 | GGAGTGGCACGTTGAGAAGAATG |
| | TTAGTTTCTGGA |
| 20 | TTACGAGTTCACTTTCTAGAAGCG |
| | GTCTGGTCAGA |
| 21 | GGTGTCCAGCAATACGAAG |
| | ATGTCCACGAAGGATC |
| 22 | CAGTGATTTCTTTAGGCAGGTCCT |
| | TGATGTCACAG |
| 23 | CGCTGCGAAGCTCCCAATTTGTAA |
| | TAAGAAAGCGT |
| 24 | ATCCTGTAGCGACTGTATGCAGCA |
| | AAACCTGAGTC |
| 25 | GCTCACAAGTAGCGAGTGTTATCA |
| | GTGCCAAGAAA |
| 26 | GCCCTCGTATGTTCCAGAAGAGCA |
| | AGGTTCTTTTA |
| 27 | GCCGTCAGGACAAGCAAAAGCAA |
| | ATTGAGTGCTAA |
| 28 | CTGATGAACAGTTTAGGTGAAACT |
| | GATCTGGCACG |
| 29 | GGACACGGGTCATCAACTACATAT |
| | GGTTGATGTTG |
| 30 | GCCTCATCCACGCACAATTCAATT |
| | AAAGGTGCTGA |
| 31 | GCACTACAAGACTACCCAATTTAG |
| | GTTCCTGGCAA |
| 32 | GTGCATTTCGCTGATTTTGGGGTC |
| | CATTATCAGAC |
| 33 | GGTTACTGCCAGTTGAATCTGAGGGT |
| | CCACCAAAC |
| 34 | TTGGGGCCGACGTTGTTTTGATCGCG |
| | CCCCACTGC |
| 35 | TTGAGTGAGAGCGGTGAACCAAGAC |
| | GCAGTATTAT |
| 36 | TGGAACGCCTTGTCCTCGAGGGAATT |
| | TAAGGTCTT |
| 37 | TCGGTAGTAGCCAATTTGGTCATCTG |
| | GACTGCTAT |
| 38 | GACTGAGATCTTTCATTTTACCGTCAC |
| | CACCACGA |
| 39 | GTCCAGCTTCTGGCCCAGTTCCTAGG |
| | TAGTAGAAA |
| 40 | CAGTTGCAACCCATATGATGCCGTCTT |
| | TGTTAGCA |
| 41 | GATTGCGGGTGCCAATGTGATCTTTT |
| | GGTGTATTC |
| 42 | TTGTTCCTTGAGGAAGTTGTAGCACG |
| | ATTGCAGCA |
| 43 | GACTGCCGCCTCTGCTCCCTTCTGCGT |
| | AGAAGCCT |
| 44 | TTCTTGAACTGTTGCGACTACGTGAT |
| | GAGGAACGA |
| 45 | CCATTCTAGCAGGAGAAGTTCCCCTA |
| | CTGCTGCCT |
| 46 | TGTCAAGCAGCAGCAAAGCAAGAGC |
| | AGCATCACCG |
| 47 | GTTGTTGGCCTTTACCAGACATTTTGC |
| | TCTCAAGC |
| 48 | CTTAGAAGCCTCAGCAGCAGATTTCT |
| | TAGTGACAG |
| 49 | CACGTCTGCCGAAAGCTTGTGTTACA |
| | TTGTATGCT |
| 50 | CTGATTAGTTCCTGGTCCCCAAAATTT |
| | CCTTGGGT |
| 51 | GGGGCAAATTGTGCAATTTGCGGCCA |
| | ATGTTTGTA |
| 52 | ACTTCCATGCCAATGCGCGACATTCC |
| | GAAGAACGC |
| 53 | AATTTGATGGCACCTGTGTAGGTCAA |
| | CCACGTTCC |
| 54 | GGCTCTGTTGGTGGGAATGTTTTGTA |
| | TGCGTCAAT |
| 55 | GCGGTAAGGCTTGAGTTTCATCAGCC |
| | TTCTTCTTT |
| 56 | CTGCAGCAGGAAGAAGAGTCACAGT |
| | TTGCTGTTTC |
| 57 | CAGCACTGCTCATGGATTGTTGCAAT |
| | TGTTTGGAG |
| 58 | CCATCTGCCTTGTGTGGTCTGCATGA |
| | GTTTAGGCC |
| 59 | GGCTCTTTCAAGTCCTCCCTAATGTTA |
| | CACACTGA |
| 60 | CTGTACACTCGATCGTACTCCGCGTG |
| | GCCTCGGTG |
| 61 | ACATTAGGGCTCTTCCATATA |
| | GGCAGCTCTCCCTA |

### Example 2

### Detection of SARS-Cov2 RNA with Single Molecule Sensitivity

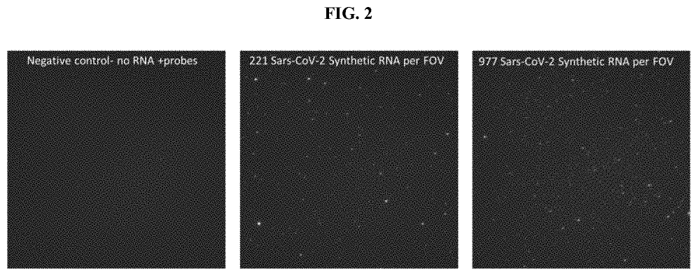

Synthetic SARS-Cov2 RNA was captured on a glass coverslip using sequences at the 3′ end of the genomic sequence. The samples were then hybridized with the 24 probes in Table 1 and imaged on the microscope. As shown in FIG. 2, each diffraction limited dot corresponded to a single mRNA molecule (FIG. 2, center and right panels). In the absence of synthetic RNA, no diffraction limited dots were observed (FIG. 2, left panel).

Interestingly, the RNA capture rate is high and exhibited linear behavior with an estimated 80% of the injected RNA injected in the flow cell captured and detected. An estimated 166,000 molecules of COVID-19 mRNA (1-type) were hybridized as estimated by calibrations based on the manufacturer IDT's provided concentration. This RNA level corresponded to approximately 221 mRNAs per a 200 um² imaging field of view (FOV) on a total flow cell surface of 3 mm².

Approximately 180 dots per FOV were detected. This indicates a detection efficiency of 81%. Increasing the number of RNA injected five-fold to 830,000 molecules, yielded an average of 977 dots per FOV. The results of this RNA concentration titration experiment demonstrated that fewer than 20% of the RNA are lost in the hybridization and capture process. In addition, the detection efficiency is linear over a range of concentrations.

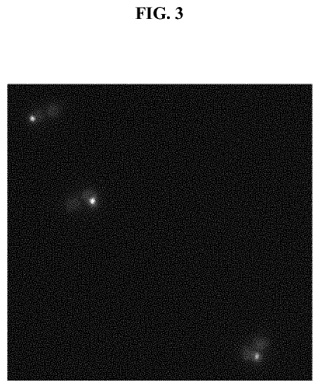

The viral RNA was shown that it can be captured on magnetic beads (FIG. 3) and detected with single molecule sensitivity with minimal background from the beads. This is particularly advantageous as functionalizing beads with the patient barcodes as described in FIG. 1 allows rapid scale up of the assay for simultaneous measurements of many patient samples.

### Example 3

### The Multiplex RNA Assay in a 96 Well Format

Barcode biotinylated oligonucleotides were conjugated to streptavidin magnetic beads. These barcodes were detected on the microscope by flowing 15 nucleotide long readout probes that were complementary to the barcoded sequence and contained a fluorophore. Because each ID bead contains thousands of oligonucleotides, the signal from these barcodes were easily detected.

Stripping and rehybridization of readout probes were performed according to the embodiments described herein.

To barcode a ID for each well in a 96 multi-well plate, two sets of oligonucleotides, corresponding to columns and rows of an individual multi-well plate, was performed such that each well was barcoded by a unique pair of oligonucleotides (1.1B). The pooled beads from all wells were decoded by iterating through the readouts that are correspond to all rows and columns.

A second round of biotin-oligonucleotide barcodes were added to the streptavidin magnetic beads, allowing the “plate” barcodes (1.4) to be conjugated to the pooled beads from the first 96 well plates.

The barcodes from the first and second of labeling were detected by sequential hybridization.

### Example 4

### RNA Probes Used for Simultaneous Detection of SARS-CoV2 and Influenza

Coinfection of COVID-19 and influenza is a major unexplored but potential serious clinical problem. Additional probe sets targeting Influenza A&B, enterovirus, hMPV, Adenovirus, Parainfluenza virus 1-4, and Rhinovirus were multiplexed.

A unique capture sequence for each infectious agent was functionalized to a distinct bead that can be multiplexed with the SARS-Cov2 beads in the same patient samples.

### Example 5

### Detecting Multiple Protein Targets Using Different Antibodies the Same Patient Samples

Multiple antibodies were measured from the same patient sample by using the same magnetic beads and workflow for RNA (1.1B) and using antigens as capture reagents.

The antibody ID beads were mixed with RNA ID beads and imaged in the same experiment. The ability to measure both antibodies and viral RNA is essential for vaccine and drug development. This technology was applied to blood samples as well as oral swabs.

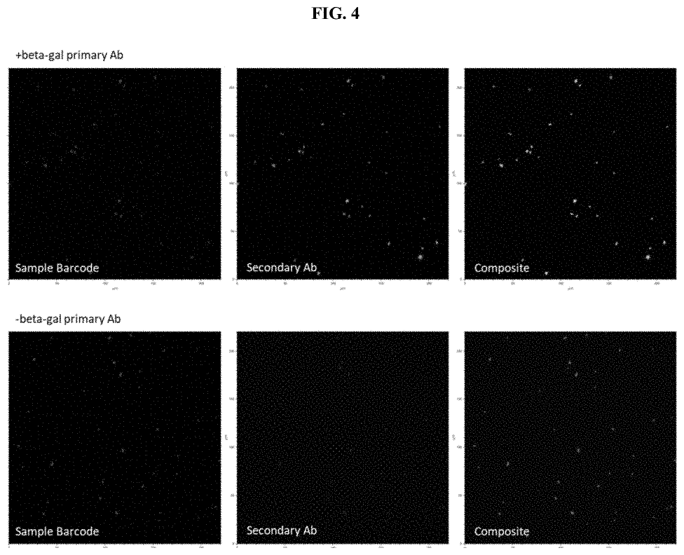

Antibodies in the sample were captured by antigens on beads. Biotinylated β-galactosidase was conjugated to streptavidin functionalized beads, along with a barcode oligo and a capture sequence that allowed the beads to attach to glass coverslips (1.1B and FIG. 4). In the presence of an anti-β-galactosidase antibody, clear fluorescence signals are observed when the beads were hybridized with a Cy3 labeled secondary antibody against the β-galactosidase antibody (FIG. 4).

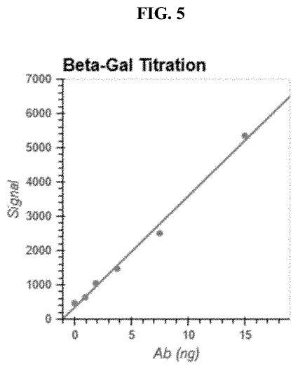

To measure the sensitivity of the assay, a concentration titration of β-galactosidase antibodies was performed (FIG. 5). Using between 1 ng to 15 ng of antibodies, a robust signal was observed at 1 ng, and a linear increase in fluorescence signal was observed as concentrations of antibodies increased.

### Example 6

### Detection of IgG Antibody in Saliva Using Spike Protein Labeled Beads

The methods disclosed herein, measured the presence of IgG antibodies in the saliva collected from patients with COVID-19 symptoms. Patient ID beads were functionalized with either spike protein or receptor binding domain (RBD)-biotin, or biotinylated β-galactosidase as a negative control.

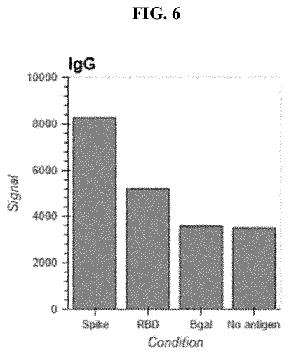

The COVID-19 symptom positive patient sample was diluted in Tris-EDTA buffer and incubated with all three beads for 5 hours, and then labeled with human anti-IgG secondary antibody. The sample showed high reactivity to both the spike protein and RBD, with minimal signal for the negative control beta-gal beads and blank beads, which both show signal at the background level (FIG. 6).

### Example 7

### Integration of Antibody Tests with Nucleic Acid Tests

Oligo-labeled secondary antibodies for IgG, IgM, or IgA were used instead of directly labelling secondary antibodies. The oligo-antibodies allowed sequential hybridization to readout the abundance of each of the isotypes by hybridizing a readout probe against the oligonucleotides on the antibodies. As an example, beads conjugated with spike protein captured IgG, IgM, and IgA antibodies from the patient sample. The conjugated beads were mixed with RNA capturing beads directed to capture SARS-CoV2 RNA. Because the probe sets that hybridizes against the SARS-CoV2 RNA on the RNA capturing beads had a different readout sequence, the identity of the beads were distinguished even when beads that capture different molecules are pooled together. Patient samples were incubated with both RNA and antibody capturing beads together or separately before they are pooled for imaging.

The panel of antibodies and proteins included cytokines, interleukins and other inflammation analytes. Commercially available ELISA assays measure these proteins one at a time. All these analytes from the same sample can be multiplexed. Many disease biomarkers were in the serum and even in saliva presumably due to lysed circulating cells and exosomes.

### Example 8

### Ratio-Metric Method for Efficient Barcoding of Magnetic Beads

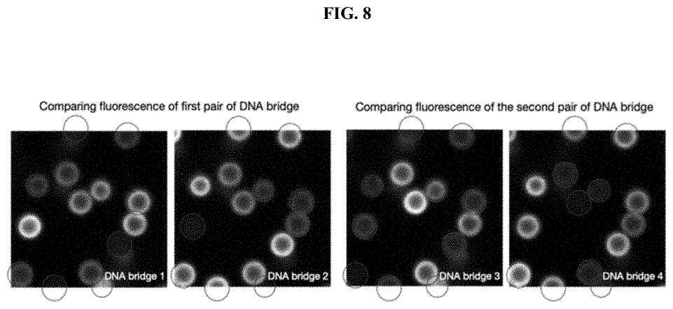

We designed oligonucleotide handles that can be conjugated to carboxyl coated magnetic beads, and DNA bridges that can bind to handles with 5′ overhang that can be read out by FISH. To barcode beads, we developed an easy to operate ratiometric scheme that uses two pairs of DNA bridge ratio to finally give 96 combinations for one handle. Combining three handles together gives us 96³ possibilities, enough for ˜10,000 patient samples with ˜100 targeting analytes. FIG. 8 shows DNA bridge readouts when performing sequential hybridization.

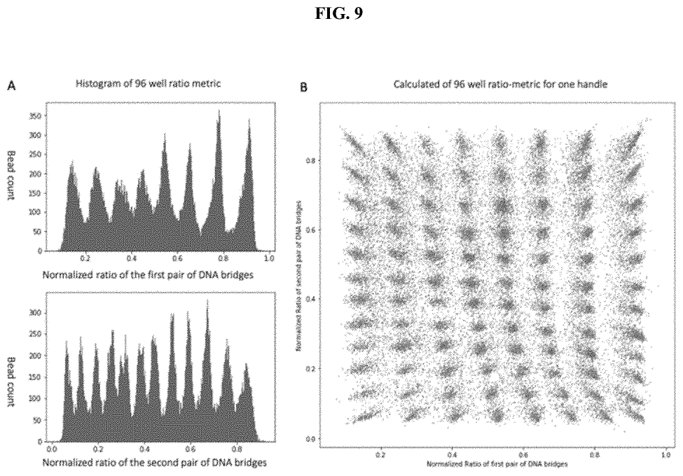

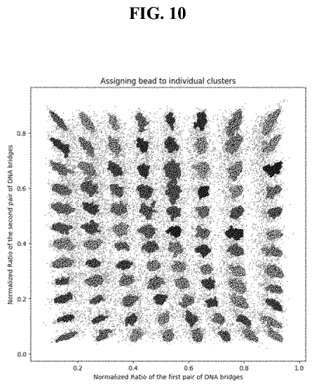

Decoding was performed as shown in FIG. 9 and FIG. 10 and demonstrated the decoding of the ratiometric barcoding using intensity ratios between DNA bridge pair.

### Example 9

### Quantification of Analyte Abundance in Fluid Samples by Imaging

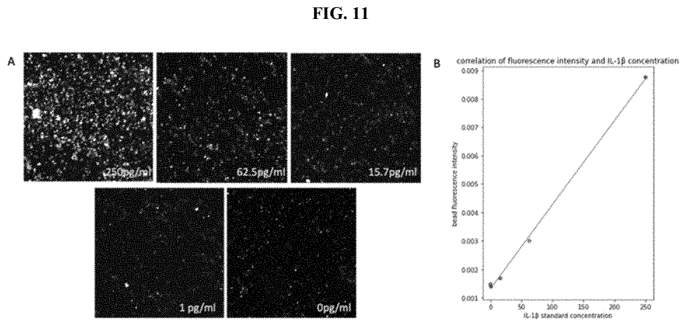

The beads were assayed to quantify the concentration of soluble protein (analyte) in fluid samples. Carboxyl magnetic beads were conjugated with IL-1b capture antibody and oligonucleotide handles at the same time. Human IL-1b was diluted in PBS solution into 250 pg/ml, 62.5 pg/ml, 15.7 pg/ml, 1 pg/ml, 0 pg/ml. These standard serial solutions were incubated with beads, then incubated with biotinylated detection antibodies to form a sandwich structure. Alexa647 conjugated streptavidin was used to bind to biotin on the detection antibody for quantification. Beads were then captured on separated spots on glass slides for imaging. FIG. 11 shows the fluorescence of beads under a fluorescent microscope.

### REFERENCES

The following references are incorporate by their entirety.
Lin Yu et al. Rapid Detection of COVID-19 Coronavirus Using a Reverse Transcriptional Loop-Mediated Isothermal Amplification (RT-LAMP) Diagnostic Platform. Clinical Chemistry, Volume 66, issue 7, July 2020, Pages 975-977.
Jiran Li and Peter B. Lillehoj. Microfluidic Magneto Immunosensor for Rapid, High Sensitivity Measurements of SARS-CoV-2 Nucleocapsid Protein in Serum. ACS Sens. 2021, 6, 3, 1270-1278.
Rebeca M. Torrente-Rodriguez et al. SARS-CoV-2 RapidPlex: A Graphene-Based Multiplexed Telemedicine Platform for Rapid and Low-Cost COVID-19 Diagnosis and Monitoring. Volume 3, Issue 6, 2 Dec. 2020, Pages 1981-1998.
Lucira Covid-19 All-In-One Test Kit. See 2nyvwd1bf4ct4f787m3leist-wpengine.netdna-ssl.com/wp-content/uploads/2020/11/Lucira-HCP-Instructions-For-Use-IFU.pdf

What is claimed is:

1. A method for detecting one or more target analytes in one or more samples comprising:
providing one or more samples;
contacting each sample with one or more primary affinity reagents, wherein each primary affinity reagent specifically binds to one or more target analytes in the one or more samples, under conditions that permit binding;
contacting one or more ID reagents to the one or more target analytes, wherein each ID reagent comprises:
one or more ID barcodes; and
one or more capture reagents capable of binding to the one or more target analytes or one or more primary affinity regents;
pooling the primary affinity reagents or ID reagents bound to the one or more target analytes; and
detecting the one or more target analytes and the one or more ID barcodes on each ID reagent or primary affinity reagent.

2. A method for detecting one or more target analytes in one or more biological samples comprising:
providing one or more samples;
contacting each sample with one or more primary affinity reagents, wherein each primary affinity reagent binds one or more target analytes in the one or more samples, under conditions that permit binding;
contacting the one or more samples with one or more ID reagents to one or more target analytes, wherein each ID reagent comprises:
one or more capture reagents, each reagent specific for one or more target analytes; and
optionally, at least two or more oligonucleotide handles, wherein one of the oligonucleotide handles identifies a specific primary affinity reagent, and wherein one or more of the oligonucleotide handles identify a sample; and
capturing the primary affinity reagent or ID reagents on a substrate for visualization; and
detecting one or more target analytes, a handle identifying a specific primary affinity reagent, and one or more handles identifying the sample on each primary affinity reagent or ID reagent.

3. A method for detecting one or more target analytes in one or more samples comprising:
providing one or more samples;
contacting each sample with one or more primary affinity reagents, wherein each primary affinity reagent binds one or more target analytes in the one or more samples, under conditions that permit binding;
contacting the one or more samples with one or more ID reagents, wherein each ID reagent comprises:
one or more capture reagents, each capture reagent specific for one or more target analytes; and
optionally, one or more oligonucleotide handles;
capturing the one or more primary affinity reagents or one or more ID reagents on a substrate for visualization; and
detecting one or more target analytes, a handle identifying a specific primary affinity reagent, one or more handles identifying the sample on each primary affinity reagent, ID reagent, or any combination thereof.

4. (canceled)

5. (canceled)

6. (canceled)

7. (canceled)

8. A method for diagnosing a subject by detecting one or more target analytes according to claim 1.

9. (canceled)

10. (canceled)

11. The method of claim 1, wherein the one or more samples are from patients suspected of having SARS-COVID-2.

12. The method of claim 1, wherein the target analytes in the sample are selected from proteins, modified proteins, transcripts, RNA, DNA loci, exogenous proteins, exogenous nucleic acids, hormones, carbohydrates, small molecules, biologically active molecules, and combinations thereof.

13. (canceled)

14. (canceled)

15. (canceled)

16. The method of claim 1, wherein each primary affinity reagent and each capture reagent comprises a specific oligonucleotide, protein, antibody, small molecule, or combination thereof to target nucleic acids, proteins and other biological molecules in the sample.

17. The method of claim 1, wherein the capture reagent comprises an oligonucleotide, protein, antibody, small molecule, or combination thereof.

18. The method of claim 17, wherein the capture reagent is capable of binding to the target analytes or primary affinity reagent.

19. (canceled)

20. The method of claim 1, further comprising isolating the one or more ID reagents.

21. The method of claim 1, wherein the one or more primary affinity reagent or ID reagents comprise patient ID barcodes or plate ID barcodes or any combination thereof.

22. The method of claim 21, wherein the primary affinity reagent or patient ID barcodes comprise oligonucleotides that identify a well in a multi-well plate.

23. (canceled)

24. (canceled)

25. (canceled)

26. (canceled)

27. The method of claim 1, wherein the ID reagents are selected from ID beads, nucleic acids, antibodies, antibody fragments, small molecules, or any combination thereof.

28. The method of claim 1, wherein one or more ID beads comprises one or more substrate binding moieties.

29. The method of claim 28, further comprising contacting the ID beads to a substrate, wherein the substrate binds the substrate binding moiety on the ID bead.

30. The method of claim 1, wherein an antibody specific to a target protein, identifies the target protein.

31. (canceled)

32. The method of claim 1, further comprising pooling IDs bead from wells of a single plate and transferring the pool into an individual well of a multi-well plate.

33. (canceled)

34. (canceled)

35. (canceled)

36. (canceled)

37. The method of claim 1, further comprising prior to contacting with the sample ID beads are pooled after hybridizing the first DNA bridge probe to the first handle, split into a multi-well plate to hybridize a second DNA bridge probe to a second handle, and split from each well in the multi-well plate to hybridize a third DNA bridge probe to a third handle.

38. (canceled)

39. The method of claim 1 wherein the primary affinity reagent comprises a protein capable of binding to one or more target nucleic acids or proteins or any combination thereof in the sample.

40. The method of claim 39, wherein the protein is an antibody or antibody fragment.

41. (canceled)

42. (canceled)

43. (canceled)

44. The method of claim 1, wherein the one or more barcodes identify a row number, column number, or any combination thereof of a well in a multi-well plate, wherein each well corresponds to one or more samples.

45. The method of claim 1, wherein the barcode binds to the ID bead through a covalent or non-covalent interaction.

46. The method of claim 1, wherein the barcode binds to the ID bead through a biotin-streptavidin interaction.

47. (canceled)

48. (canceled)

49. (canceled)

50. The method of claim 1, wherein the substrate binding moiety comprises a poly A nucleotide sequence.

51. (canceled)

52. (canceled)

53. (canceled)

54. (canceled)

55. The method of claim 2, wherein the barcodes are ratiometric.

56. The method of claim 55, wherein the ratiometric barcodes are generated by titrating one or more DNA bridge probes at different ratios for each oligonucleotide handle.

57. The method of claim 56, wherein four DNA bridges are used per oligonucleotide handle, and wherein the DNA bridges are separated into two pairs of oligonucleotides.

58. The method of claim 56, wherein the first pair of DNA bridges is titrated to a oligonucleotide handle with a first set of ratios.

59. The method of claim 56, wherein the second pair of DNA bridges is titrated to a oligonucleotide handle with second set of ratios.

60. (canceled)

61. The method of claim 1, further comprising quantifying the amount of analytes present in each sample.
